(19) United States
(12) Patent Application Publication
(10) Pub. No.: US 2014/0296566 A1
(43) Pub. Date: Oct. 2, 2014

(54) Simplified Radiosynthesis of O-[18F]Fluoromethyl Tyrosine Derivatives

(71) Applicants: Keith Andrew Nenton Graham, Berlin (DE); Selahattin Ede, Berlin (DE)

(72) Inventors: Keith Andrew Nenton Graham, Berlin (DE); Selahattin Ede, Berlin (DE)

(73) Assignee: PIRAMAL IMAGING SA, MATRAN (CH)

(21) Appl. No.: 14/240,518

(22) PCT Filed: Aug. 27, 2012
(86) PCT No.: PCT/EP2012/066618

§ 371 Date: May 7, 2014

(30) Foreign Application Priority Data
Aug. 25, 2011 (EP) 11178906.1

(51) Int. Cl. C07C 227/40

(52) U.S. Cl. CPC C07C 227/40
USPC 562/444

(57) ABSTRACT

This invention relates to the simplified radiosynthesis of O-[¹⁸F]fluoromethyl tyrosine derivatives whereby the need for purification by preparative high pressure liquid chromatographic methods (HPLC) has been eliminated.

### FIELD OF INVENTION

This invention relates to the simplified radiosynthesis of O-[¹⁸F]fluoromethyl tyrosine derivatives whereby the need for purification by preparative high pressure liquid chromatographic methods (HPLC) has been eliminated.

### BACKGROUND

The invention relates to the subject matter referred to in the claims, i.e. a simplified radiosynthesis of O-[¹⁸F]fluoromethyl tyrosine derivatives of the formula (I).

Molecular imaging has the potential to detect disease progression or therapeutic effectiveness earlier than most conventional methods in the fields of oncology, neurology and cardiology. Of the several promising molecular imaging technologies having been developed as optical imaging and MRI, PET is of particular interest for drug development because of its high sensitivity and ability to provide quantitative and kinetic data.

Positron emitting isotopes include carbon, nitrogen, and oxygen. These isotopes can replace their non-radioactive counterparts in target compounds to produce tracers that function biologically and are chemically identical to the original molecules for PET imaging. On the other hand, ¹⁸F is the most convenient labeling isotope due to its relatively long half life (109.6 min) which permits the preparation of diagnostic tracers and subsequent study of biochemical processes. In addition, its high β+ yield and low β+ energy (635 keV) are also advantageous.

Due to its short 20 minutes half-life ¹¹C containing radiotracers require an on-site cyclotron, whereas ¹⁸F PET tracers, considering a half-life of 109 minutes, allow for off-site production and regional distribution.

Radiolabeled amino acids have been explored for tumor imaging (Jager et al., J Nucl Med., 2001, 42(3), 432-45) to overcome the limitations seen for [¹⁸F]FDG. Initially, naturally occurring amino acids were labeled with carbon-11 such [¹¹C]valine, L-[¹¹C]leucine, L-[¹¹C]methionine, and structurally similar [¹⁸F]analogues. After uptake of these mainly neutral amino acids into the tumor cells predominantly via sodium independent L-type amino acid transporters, the retention of these tracers within the tumor cell is mainly due to protein synthesis. Limitations of radiolabeled naturally occurring amino acids include metabolic degradation via several pathways resulting in multiple radiolabeled metabolites which obscure the analysis of tumor uptake of the mother compound. Kinetic studies have suggested that amino acid transport rather than protein synthesis better reflect tumor proliferation. Tyrosine derivatives have been of particular interest over the last decade with the introduction of [¹⁸F]fluoroethyl tyrosine ([¹⁸F]FET) which has been shown to be effective in imaging brain tumors but not for periphery tumors (Wester et al., J. Nucl. Med. 1999, 40, 663, J. Nucl. Med. 1999, 40, 205 and J. Nucl. Med. 1999, 40, 1367). Other potentially interesting tyrosine derivatives include 3-[¹⁸F]fluoro-α-methyl tyrosine (Inoue et al., J. Nucl. Med. 1998, 39, 205), O-[¹⁸F]fluoromethyl tyrosine (Ishiwata et al., Nucl. Med. Biol., 2004, 31, 191), O-[¹⁸F]fluoropropyl tyrosine (Tang et al., Nucl. Med. Biol., 2003, 30, 733), O-[¹⁸F]fluoropropenyl tyrosine (Arstad et al., WO2007073200) and O-[¹⁸F]fluoroethyl-α-methyl tyrosine (Wang et al., Bioorg. Med. Chem. Lett., 2010, 20, 3482). These radiolabeled tyrosine derivatives all have their disadvantages, i.e. low yield and high mass dose due to the electrophilic radiofluorination method used for the production of 3-[¹⁸F]fluoro-α-methyl tyrosine. The other tyrosine derivatives either show poor uptake in cell culture, low accumulation in tumor bearing mice or relatively poor pharmacokinetics resulting in images with a high background. D-[¹⁸F]fluoromethyl tyrosine (DFMT, Tsukada et al., J. Nucl. Med., 2006, 47, 679, Tsukada et al., Eur. J. Nucl. Med. Mol. Imaging, 2006, 33, 1017 and Urakami et al., Nucl. Med. Biol., 2009, 36, 295) has recently been shown to give better images in tumor bearing mice. The reason for this could be that the D-isomer is recognised by the tumor but, in comparison to its L-isomer, not by other organs within the body and is excreted rapidly via the kidneys, thus enhancing the image quality (Tsukada et al., J. Nucl. Med., 2006, 47, 679).

The radiosyntheses of O-[¹⁸F]fluoromethyl tyrosine derivatives are typically carried out via a two-step process (Scheme 1): i) radiofluorination of a precursor (e.g. dibromomethane) to give a [¹⁸F]labeled alkylating agent (e.g. [¹⁸F]fluoromethyl bromide); ii) alkylation of tyrosine with the [¹⁸F]labeled alkylating agent following by semi-preparative HPLC purification to give the desired O-[¹⁸F]fluoromethyl tyrosine derivative. A very important technical goal in using ¹⁸F-labeled radiopharmaceuticals is the quick preparation and administration of the radioactive compound due to the fact that the ¹⁸F isotopes have a short half-life of about only 110 minutes.

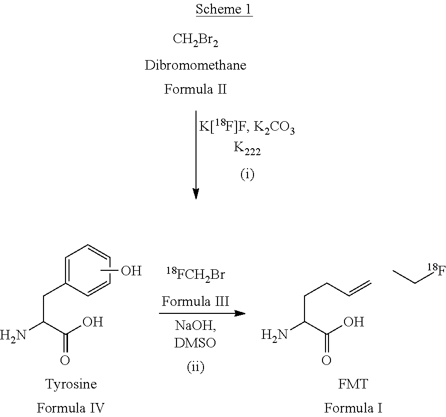

There is a continued need for novel methods for improving and simplifying the radiosyntheses of F-18 radiolabeled compounds. This present application discloses methods of synthesizing radiolabeled O-[¹⁸F]fluoromethyl tyrosine derivatives.

The synthesis of [¹¹C]methyl tyrosine and [¹⁸F]fluoroethyl tyrosine (FET) via indirect methods have been investigated by different research groups with the view to simplify the syntheses and improve the yields. The group of Iwata et al. (Appl. Radiat. Isot. 2005, 63, 55-61) simplified the radiosynthesis of [¹¹C]methyl tyrosine whereby they looked at alkylating disodium tyrosine with either [¹¹C]methyl iodide or triflate, preferably [¹¹C]methyl triflate which involves an additional step of converting the [¹¹C]methyl iodide to the [¹¹C]methyl triflate using a heated silver triflate column. They found that using a combination of a tC18 SPE and SCX SPE gave the necessary separation of the tyrosine from the radiolabeled [¹¹C]methyl tyrosine.

For the synthesis of FET, Tang et al., have looked at simplifying the synthesis by reacting the 1,2-ditosyloxyethane precursor with [¹⁸F]Fluorine isotope bound to an ammonium cation resin and eluting the desired F18-labeled alkylating agent [¹⁸F]fluoroethyl tosylate with the remaining 1,2-ditosyloxyethane into the tyrosine salt and reacting to give FET and then purify the product with a series of silica, C18 and alumina SPEs. Unfortunately, this method did not report the absence or presence of tyrosine or other impurities in the final product, and literature reports show that there are other impurities in the final solution when using this method and that this method could not trap [¹¹C]methyl tyrosine on the SPEs (Appl. Radiat. Isot. 2005, 63, 55-61). Zuhayra et al. (Bioorg. Med. Chem. 2009, 17, 7441-7448) looked at a different precursor to synthesize the [¹⁸F]-labeled alkylating agent [¹⁸F]fluoroethyl bromide and purify this by distillation. The alkylation was carried out using tyrosine, sodium methanolate in methanol as base and sodium iodide. The purification was carried out with two C18 Chromafix SPE cartridges with ammonium acetate and 5% ethanol in saline washes. Product was eluted with 10% ethanol in saline.

Müller et al. (Nuclear Medicine and Biology, 38 (2011) 653-658) described a purification method based on standard cartridges applicable to L-[¹⁸F]FET that is obtained by indirect labelling of [¹⁸F]fluoroethyltosylate with L-tyrosine. Purification method consists into first passing L-[¹⁸F]FET onto a SCX cartridge (Silica ion exchange solid phase extraction, Merck), secondly passing the L-[¹⁸F]FET onto a HRX cartridge (Polystyrene-Divinylbenzene-Copolymer solid phase extraction, Macherey-Nagel) and thirdly passing the L-[¹⁸F]FET onto two SCX cartridges. Kryptofix is removed from the final product by the HRX cartridge followed by two SCX cartridges. Residual solvents such as ethanol, acetonitrile and acetone were found by gas chromatography.

### PROBLEM TO BE SOLVED BY THE INVENTION AND ITS SOLUTION

Despite the aforementioned advances in identifying DFMT as a suitable tyrosine derivative for tumor imaging and specifically for the imaging of cancer, there remains a need to improve and simplify the radiosynthesis of such O-[¹⁸F]fluoromethyl tyrosine derivatives. The current methods to synthesis such O-[¹⁸F]fluoromethyl tyrosine derivatives use the method developed by Iwata et al. (J. Label. Compd. Radiopharm. 2003, 46, 555-566) and have been used by others for the synthesis of DFMT (Tsukada et al., J. Nucl. Med., 2006, 47, 679-688) and follows the same method outlined in Scheme 1 with purification of the final product by semi-preparative HPLC followed by concentration of this final solution to allow the tracer to be used in the in vivo settings. This method is relatively complicated as a number of steps are necessary to complete the synthesis: 1) radiofluorination of the precursor; 2) distillation of the [¹⁸F]fluoromethyl bromide and optionally converting it to [¹⁸F]fluoromethyl triflate (Iwate et al., J. Label. Compd. Radiopharm. 2003, 46, 555-566); 3) alkylation of the tyrosine with [¹⁸F]fluoromethyl bromide; 4) purification of the desired product by semi-preparative HPLC; 5) concentration of the HPLC fraction to remove non-injectable compounds, i.e. ethanoic acid and 6) reformulation to allow for the said tyrosine derivative to be used in vivo. This process is rather long, needing multiple transformations/steps and requires a number of dedicated pieces of equipment, i.e. automated synthesis module(s), rotary evaporator, which require space and additional shielding to protect the operator(s).

We found that the final O-[¹⁸F]fluoromethyl tyrosine could be purified using a surprisingly simple solid phase extraction (SPE) cartridge(s) instead of using expensive, cumbersome and time-consuming HPLC purification methods and their additional SPE step needed to create a formulated solution ready for injection by removing toxic and potential toxic solvents and additives used in the liquid phase of the HPLC purification. The simple method requires the alkylation reaction method of O-[¹⁸F]fluoromethyl tyrosine, O-[¹⁸F]fluoromethyl bromide, tyrosine, organic solvent (e.g. DMSO) and base (e.g. sodium hydroxide) to be diluted with an acidic water solution and passed through different SPEs, e.g. strong cation exchange (SCX) SPE or hydrophilic-lipophilic balance (HLB) SPEs. These SPEs trapped the desired O-[¹⁸F]fluoromethyl tyrosine derivatives and the other by-product, starting materials could be removed with a simple washing step, allowing the desired product to be eluted from the SPE in high radiochemical and chemical purity.

### SUMMARY

The invention relates to the methods referred to in the claims for the simplified radiosynthesis of O-[¹⁸F]fluoromethyl tyrosine derivatives.

### DESCRIPTION

In a first aspect, the invention is directed to methods for the purification of compound of formula (I)

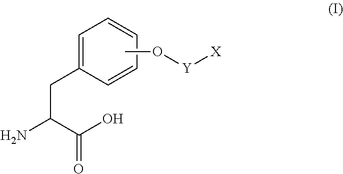

wherein
   X is a Fluorine atom (F);
   Y is CH₂, CHD, or CD₂; and
   D stands for Deuterium

comprising the step of
Purification of compound of formula (I) by solid-phase-extraction (SPE) conducted with
   one SPE cartridge wherein the solid phase of the cartridge is a cation exchange resin or
   two to four SPE cartridges wherein the solid phase of the cartridge is a polymer based resin.

Preferably, the step comprises
Purification of compound of formula (I) by solid-phase-extraction (SPE) conducted with
   one SPE cartridge wherein the solid phase is a strong cation exchange resin selected from SCX (Strong Cationic Exchanger) or
   two to four SPE cartridges wherein the solid phase is a polymer based resin polymeric water-wettable reversed-phase sorbent selected from HLB (Hydrophilic-Lipophilic Balance).

More preferably, the step comprises
Purification of compound of formula (I) by solid-phase-extraction (SPE) conducted with
   one SCX SPE cartridge or
   two to four HLB SPE cartridges.

Formula (I) encompasses single isomers, and enantiomers, mixtures thereof and pharmaceutically acceptable salts thereof.

Preferred Features:

Preferably, X is a Fluorine atom (F) selected from [¹⁸F] or [¹⁹F] Fluorine isotope. More preferably X is a [¹⁸F] Fluorine isotope.

Preferably, Y is CH₂ or CD₂. More preferably, Y is CH₂.

Preferably, compounds of formula (I) are compounds of formula (I-D) or (I-L).

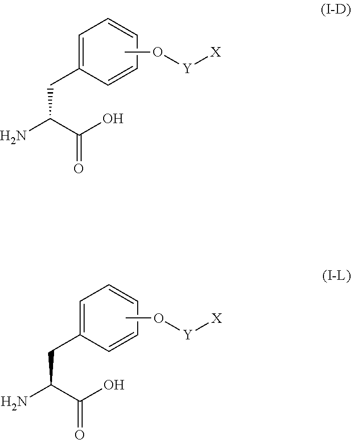

Formula (I-D) and (I-L) encompass single isomers, and enantiomers, mixtures thereof and pharmaceutically acceptable salts thereof.

More preferably, compounds of formula (I) are compounds of formula (I-D).

Even more preferably, compounds of formula (I) are [¹⁸F]DFMT standing for (R)-2-Amino-3-(4-[¹⁸F]fluoromethoxy-phenyl)-propionic acid, DFMT standing for (R)-2-Amino-3-(4-fluoromethoxy-phenyl)-propionic acid, [¹⁸F]Deuterio-DFMT standing for (R)-2-Amino-3-(4-[¹⁸F]fluorodideuteriomethoxy-phenyl)-propionic acid or Deuterio-DFMT standing for (R)-2-Amino-3-(4-fluorodideuteriomethoxy-phenyl)-propionic acid.

More preferably, compounds of formula (I) are compounds of formula (I-L).

Even more preferably, compounds of formula (I) are [¹⁸F]FMT standing for (S)-2-Amino-3-(4-[¹⁸F]fluoromethoxy-phenyl)-propionic acid, FMT standing for (S)-2-Amino-3-(4-fluoromethoxy-phenyl)-propionic acid, [¹⁸F]Deuterio-FMT standing for (S)-2-Amino-3-(4-[¹⁸F]fluorodideuteriomethoxy-phenyl)-propionic acid or Deuterio-FMT standing for (S)-2-Amino-3-(4-fluorodideuteriomethoxy-phenyl)-propionic acid.

Solid-phase-extraction (SPE) consists of trapping small molecules such as compounds of formula (I) on solid phase of a cartridge or column and then eluting the small molecules such as compounds of formula I with aqueous buffer wherein the solid phase is a cation exchange resin or a polymer based resin. Side products, prosthetic group additives and potentially toxic organic solvents are removed from the eluted solution.

Preferably, the solid phase is a cation exchange resin that is a weak, medium or strong cation exchange resin, more preferably the cation exchange resin is a medium or strong cation exchange resin, even more preferably the cation exchange resin is a strong cation exchange resin.

More preferably, the cation exchange resin contained in the SPE cartridge is of the range from about 0.1 to about 2 g, preferably about 0.2 to about 1.5 g, more preferably about 0.8 to about 1.5 g, even more preferably about 1 g.

Even more preferably, the solid phase is a strong cation exchange resin defined as SCX (Strong Cationic Exchanger).

SCX SPE cartridge is a cartridge containing 1 g of SCX resin.

Purification occurs by solid-phase-extraction using a single SCX SPE cartridge.

Preferably, the solid phase is a polymer based resin. Preferably, the polymer based resin is polymeric water-wettable reversed-phase sorbent selected from HLB (Hydrophilic-Lipophilic Balance).

More preferably, the polymeric water-wettable reversed-phase sorbent contained in the SPE cartridge is of the range from about 0.1 to about 2 g, preferably about 0.2 to about 1.5 g, more preferably about 0.8 to about 1.5 g, even more preferably about 0.9 g.

HLB SPE cartridge is a cartridge containing 0.9 g of HLB resin.

Purification occurs by solid-phase-extraction using two to four HLB SPE cartridges wherein HLB SPE cartridges are mounted in line.

In a first embodiment, the invention is directed to methods wherein the compound of formula (I) is defined such as Fluorine atom (F) is an ¹⁸F isotope. Preferably, the invention is directed to methods wherein the compound of formula (I) is a compound of formula (I-D). More preferably, the invention is directed to methods wherein the compound of formula (I) is [¹⁸F]DFMT standing for (R)-2-Amino-3-(4-[¹⁸F]fluoromethoxy-phenyl)-propionic acid.

In a second embodiment, the invention is directed to methods wherein the compound of formula (I) is defined such as Fluorine atom (F) is an ¹⁹F isotope. Preferably, the invention is directed to methods wherein the compound of formula (I) is a compound of formula (I-D). More preferably, the invention is directed to methods wherein the compound of formula (I) is DFMT standing for (R)-2-Amino-3-(4-fluoromethoxy-phenyl)-propionic acid.

Preferably, the method for the purification of compound of formula (I)

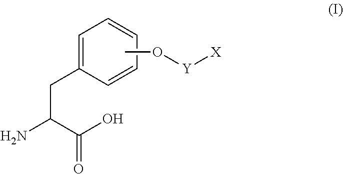

wherein
   X is a Fluorine atom (F);
   Y is CH₂, CHD, or CD₂; and
   D stands for Deuterium

comprises the step of
Purification of compound of formula (I) by solid-phase-extraction conducted with one SCX SPE cartridge.

Preferably, the method for the purification of compound of formula (I)

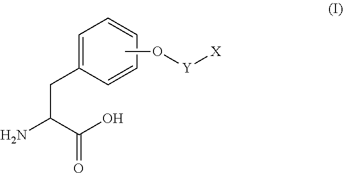

wherein
   X is a Fluorine atom (F);
   Y is CH₂, CHD, or CD₂; and
   D stands for Deuterium comprises the step of
Purification of compound of formula (I) by solid-phase-extraction conducted with two to four HLB SPE cartridges.

Embodiments and preferred features can be combined together and are within the scope of the invention.

For the formulation of the above mentioned PET tracers of formula I, the solution eluted from the said SPE or SPEs can be pH adjusted and sterile filtered or the eluting solution volume can be increased and sterile-filtered.

In a second aspect, the invention is directed to methods for obtaining purified compound of formula (I) suitable for injection into patients comprising the step of
Indirect fluoro-labeling or direct fluoro-labeling step for obtaining compound of formula (I)

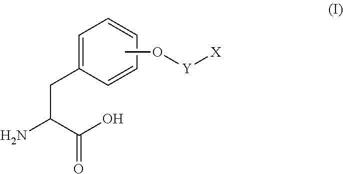

wherein
   X is a Fluorine atom (F);
   Y is CH₂, CHD, or CD₂; and
   D stands for Deuterium

and
Purification of compound of formula (I) by solid-phase-extraction (SPE) conducted with
   one SPE cartridge wherein the solid phase of the cartridge is a cation exchange resin or
   two to four SPE cartridges wherein the solid phase of the cartridge is a polymer based resin.

Preferably, the step comprises
Purification of compound of formula (I) by solid-phase-extraction (SPE) conducted with
   one SPE cartridge wherein the solid phase is a strong cation exchange resin selected from SCX (Strong Cationic Exchanger) or
   two to four SPE cartridges wherein the solid phase is a polymer based resin polymeric water-wettable reversed-phase sorbent selected from HLB (Hydrophilic-Lipophilic Balance).

More preferably, the step comprises
Purification of compound of formula (I) by solid-phase-extraction (SPE) conducted with
   one SCX SPE cartridge or
   two to four HLB SPE cartridges.

Even more preferably, the step comprises
Purification of compound of formula (I) by solid-phase-extraction (SPE) conducted with one SCX SPE cartridge.

Even more preferably, the step comprises
Indirect fluoro-labeling step for obtaining compound of formula (I) and
Purification of compound of formula (I) by solid-phase-extraction (SPE) conducted with one SCX SPE cartridge.

Even more preferably, the step comprises
Purification of compound of formula (I) by solid-phase-extraction (SPE) conducted with two to four HLB SPE cartridges.

Even more preferably, the step comprises
Indirect fluoro-labeling step for obtaining compound of formula (I) and
Purification of compound of formula (I) by solid-phase-extraction (SPE) conducted with two to four HLB SPE cartridges.

Formula (I) encompasses single isomers, and enantiomers, mixtures thereof and pharmaceutically acceptable salts thereof.

Preferred Features:

Preferably, X is a Fluorine atom (F) selected from [¹⁸F] or [¹⁹F] Fluorine isotope. More preferably X is a [¹⁸F] Fluorine isotope.

Preferably, Y is CH₂ or CD₂. More preferably, Y is CH₂.

Preferably, the methods for obtaining compound of formula (I) are indirect fluoro-labeling methods or direct fluoro-labeling methods.

Indirect fluoro-labeling methods are well known in the art.

The indirect fluoro-labeling method comprises the steps
Coupling compound of Formula (II) with Fluorine atom (F) containing moiety for obtaining compound of Formula (III)
Coupling compound of Formula (III) with a compound of formula (IV) for obtaining compound of Formula (I),
Optionally converting obtained compound into a pharmaceutically acceptable salts of inorganic or organic acids thereof, hydrates, complexes, esters, amides, and solvates thereof.

Preferably, the methods are indirect labeling methods for obtaining compound of formula (I).

The indirect fluoro-labeling method comprises the steps
Coupling compound of Formula (II) with Fluorine atom (F) containing moiety wherein the Fluorine atom (F) containing moiety comprises [¹⁹F] for obtaining compound of Formula (III)
Coupling compound of Formula (III) with a compound of formula (IV) for obtaining compound of Formula (I)
Purifying a compound of Formula (I) using solid phase extraction cartridges to remove compounds of Formulae (II), (III), (IV), additives and potentially toxic organic solvents
Optionally converting obtained compound into a pharmaceutically acceptable salts of inorganic or organic acids thereof, hydrates, complexes, esters, amides, and solvates thereof.

Preferably, the methods are indirect labeling methods for obtaining compound of formula (I).

The indirect fluoro-labeling method comprises the steps
Coupling compound of Formula (II) with Fluorine atom (F) containing moiety wherein the Fluorine atom (F) containing moiety comprises [¹⁸F]-Fluorine for obtaining compound of Formula (III)
Coupling compound of Formula (III) with a compound of formula (IV) for obtaining compound of Formula (I)
Purifying a compound of Formula (I) using solid phase extraction cartridges to remove compounds of Formulae (II), (III), (IV), additives and potentially toxic organic solvents
Optionally converting obtained compound into a pharmaceutically acceptable salts of inorganic or organic acids thereof, hydrates, complexes, esters, amides, and solvates thereof.

Compounds of formula (II) are well known suitable precursors for the synthesis of known F-18 labeled prosthetic groups (Zhang et al., Bioog. Med. Chem., 2005, 13, 1811-1818).

Preferably, compounds of formula (II) is

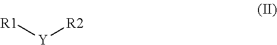

wherein
R1 is a leaving group selected from the group of halogen and sulfonate, wherein Halogen is chloro, bromo or iodo, and sulfonate is mesylate, toyslate, triflate or nosylate;
R2 is a leaving group selected from the group of halogen and sulfonate, wherein Halogen is chloro, bromo or iodo, and sulfonate is mesylate, toyslate, triflate or nosylate;
Y is CHD or CD₂ and
D stands for Deuterium.

Non-limiting examples of compounds of Formula (II) known to those skilled in the art are (Eaborn and Stanczyk, J. Chem. Soc. Perkin Trans 2, 1991, 471-473; Bothner-By et al., J. Am. Chem. Soc., 1987, 109, 4180-4184; Takaya et al., J. Org. Chem., 1981, 46, 2846-2854):
deuterated dibromomethane (CD₂Br₂), monodeuteriodibromomethane (CHDBr₂),
deuterated diiodomethane (CD₂I₂), monodeuteriodiiodomethane (CHDI₂)

Preferably, compound of Formula (II) is deuterated dibromomethane (CD₂Br₂).

Compounds of formula (III) are well known suitable F-18 labeled prosthetic groups (Zhang et al., Bioog. Med. Chem., 2005, 13, 1811-1818).

Preferably, compounds of formula (III) is

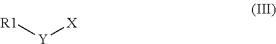

wherein
R1 is a leaving group selected from the group of halogen and sulfonate, wherein Halogen is chloro, bromo or iodo, and sulfonate is mesylate, toyslate, triflate or nosylate;
X is Fluorine atom (F) preferably Fluorine atom (F) is an ¹⁸F isotope;
Y is CHD or CD₂ and
D stands for Deuterium.

Non-limiting examples of compounds of Formula (III) known to those skilled in the art are:
deuterated bromofluoromethane (FCD₂Br), deuterated bromo[¹⁸F]fluoromethane ([¹⁸F]FCD₂Br), monodeuteriobromofluoromethane (FCHDBr), monodeuterio-bromo[¹⁸F]fluoromethane ([¹⁸F]FCHDBr), deuterated fluoroiodomethane (FCD₂I), deuterated [¹⁸F]fluoroiodomethane ([¹⁸F]FCD₂I), monodeuteriofluoroiodomethane (FCHDI), monodeuterio[¹⁸F]fluoroiodomethane ([¹⁸F]FCHDI),

Preferably, compound of Formula (III) is deuterated bromo[¹⁸F]fluoromethane ([¹⁸F]FCD₂Br).

Compounds of formula (IV) are well known D- or L-Tyrosine or mixtures thereof and/or salts thereof suitable as precursor for the indirect labelling.

Non-limiting examples of compounds of Formula (IV) known to those skilled in the art are:
L-Tyrosine, D-Tyrosine and mixtures thereof.
Salts of L-Tyrosine, D-Tyrosine and mixtures thereof

Preferably, compound of Formula (IV) is D-Tyrosine.

Compounds of formula (I) is preferably compound of formula (I-D) or (I-L).

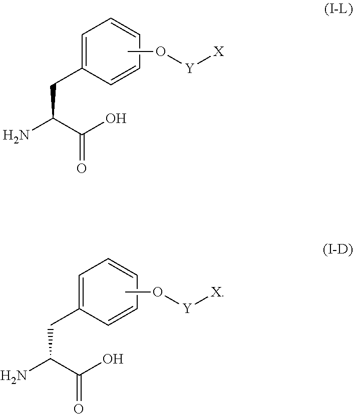

Formula (I-D) and (I-L) encompass single isomers, and enantiomers, mixtures thereof and pharmaceutically acceptable salts thereof.

More preferably, compounds of formula (I) are compounds of formula (I-D).

Even more preferably, compounds of formula (I) are [¹⁸F]DFMT standing for (R)-2-Amino-3-(4-[¹⁸F]fluoromethoxy-phenyl)-propionic acid, DFMT standing for (R)-2-Amino-3-(4-fluoromethoxy-phenyl)-propionic acid, [¹⁸F]Deuterio-DFMT standing for (R)-2-Amino-3-(4-[¹⁸F]fluorodideuteriomethoxy-phenyl)-propionic acid or Deuterio-DFMT standing for (R)-2-Amino-3-(4-fluorodideuteriomethoxy-phenyl)-propionic acid.

More preferably, compounds of formula (I) are compounds of formula (I-L).

Even more preferably, compounds of formula (I) are [¹⁸F]FMT standing for (S)-2-Amino-3-(4-[¹⁸F]fluoromethoxy-phenyl)-propionic acid, FMT standing for (S)-2-Amino-3-(4-fluoromethoxy-phenyl)-propionic acid, [¹⁸F]Deuterio-FMT standing for (S)-2-Amino-3-(4-[¹⁸F]fluorodideuteriomethoxy-phenyl)-propionic acid or Deuterio-FMT standing for (S)-2-Amino-3-(4-fluorodideuteriomethoxy-phenyl)-propionic acid.

In a first embodiment, the invention is directed to methods wherein the compound of formula (I) is defined such as Fluorine atom (F) is an ¹⁸F isotope. Preferably, the invention is directed to methods wherein the compound of formula (I) is a compound of formula (I-D). More preferably, the invention is directed to methods wherein the compound of formula (I) is [¹⁸F]DFMT standing for (R)-2-Amino-3-(4-[¹⁸F]fluoromethoxy-phenyl)-propionic acid.

In a second embodiment, the invention is directed to methods wherein the compound of formula (I) is defined such as Fluorine atom (F) is an ¹⁹F isotope. Preferably, the invention is directed to methods wherein the compound of formula (I) is a compound of formula (I-D). More preferably, the invention is directed to methods wherein the compound of formula (I) is DFMT standing for (R)-2-Amino-3-(4-fluoromethoxy-phenyl)-propionic acid.

Embodiments and preferred features can be combined together and are within the scope of the invention. Embodiments and preferred features of the first aspect are herein enclosed.

In a preferred embodiment, the method is carried out by use of a module (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated synthesis. More preferably, the process is carried out by use of an one-pot module.

The reagents, solvents and conditions which can be used for this fluorination are common and well-known to the skilled person in the field. See, e.g., J. Fluorine Chem., 27 (1985):177-191.

Preferably, the solvents used in the present method is DMF, DMSO, acetronitrile, DMA, or mixture thereof, preferably the solvent is acetonitrile.

The reagents, solvents and conditions which can be used for the alkylation are common and well-known to the skilled person in the field. See, e.g., Wester et al., J. Nucl. Med. 1999, 40, 663.

Preferably, the solvents used in the present method is DMF, DMSO, acetronitrile, DMA, or mixture thereof, preferably the solvent is DMSO.

More preferably, the Fluorine atom (F) containing moiety comprising ¹⁸F can be chelated complexes known to those skilled in the art, e.g. 4,7,13,16,21,24-Hexaoxa-1,10-diazabicyclo[8.8.8]-hexacosane K¹⁸F (crown ether salt Kryptofix K¹⁸F), 18-crown-6 ether salt K¹⁸F, K¹⁸F, H¹⁸F, KH¹⁸F₂, Rb¹⁸F, Cs¹⁸F, Na¹⁸F, or tetraalkylammonium salts of ¹⁸F known to those skilled in the art, e.g. [¹⁸F] tetrabutylammonium fluoride, or tetraalkylphosphonium salts of ¹⁸F known to those skilled in the art, e.g. [¹⁸F] tetrabutylphosphonium fluoride. Most preferably, the Fluorine atom (F) containing moiety is Cs¹⁸F, K¹⁸F, H¹⁸F, or KH¹⁸F₂.

More preferably, Fluorine atom (F) containing moiety comprises ¹⁹F. Even more preferably, the Fluorine atom (F) containing moiety is 4,7,13,16,21,24-Hexaoxa-1,10-diazabicyclo[8.8.8]-hexacosane KF (crownether salt Kryptofix KF), 1,4,7,10,13,16-hexaoxacyclooctadecane KF, KF, tetrabutylammonium fluoride, tetrabutylammonium dihydrogen trifluoride.

In a third aspect, the invention is directed to a composition comprising compounds of the formula (I) obtained by the methods of the first aspect or the second aspect and pharmaceutically acceptable carrier or diluent.

The person skilled in the art is familiar with auxiliaries, vehicles, excipients, diluents, carriers or adjuvants which are suitable for the desired pharmaceutical formulations, preparations or compositions on account of his/her expert knowledge.

The administration of the compounds, pharmaceutical compositions or combinations according to the invention is performed in any of the generally accepted modes of administration available in the art. Intravenous deliveries are preferred.

Definitions

The terms used in the present invention are defined below but are not limiting the invention's scope.

If chiral centers or other forms of isomeric centers are present in a compound according to the present invention, all forms of such stereoisomers, including enantiomers and diastereoisomers, are intended to be covered herein. Compounds containing chiral centers may be used as racemic mixture or as an enantiomerically enriched mixture or as a diastereomeric mixture or as a diastereomerically enriched mixture, or these isomeric mixtures may be separated using well-known techniques, and an individual stereoisomer maybe used alone. In cases wherein compounds may exist in tautomeric forms, such as keto-enol tautomers, each tautomeric form is contemplated as being included within this invention whether existing in equilibrium or predominantly in one form.

In the context of the present invention, preferred salts are pharmaceutically acceptable salts of the compounds according to the invention. The invention also comprises salts which for their part are not suitable for pharmaceutical applications, but which can be used, for example, for isolating or purifying the compounds according to the invention.

Pharmaceutically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, formic acid, acetic acid, trifluoroacetic acid, propionic acid, lactic acid, tartaric acid, malic acid, citric acid, fumaric acid, maleic acid and benzoic acid.

Pharmaceutically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solid-phase extraction (SPE) is an extraction method that uses a solid phase and a liquid phase to isolate analytes or products of a pre-defined type, e.g. lipophilic, hydrophilic, basic, acidic, from a solution containing different species. The general method is to load a solution onto the SPE phase and trap the desired analyte or product, wash away undesired components. Then the desired analyte or product is eluted with a different solvent or solution and collected. Solid-phase extractions use the similar types of stationary phases that are used in liquid chromatography columns. The stationary phase is usually contained in a glass or plastic column above a frit or glass wool. Commercial SPE cartridges have 1-10 mL capacities and are discarded after use. Non-limiting examples of the stationary solid phases are: silica gel, modified silica gel, alumina, resins, polymers, co-polymers or mixtures or layers thereof. In a more preferred embodiment, the stationary phase is selected from the group comprising silica, alumina A, alumina B, alumina N, magnesium silicate, magnesium oxide, zirconium oxide, C30, C18, tC18, C8, C4, C2, tC2, amino propyl (NH2), cyano propyl (CN), diol, hydroxyapatite, cellulose, graphitized carbon, weak cation exchange, medium cation exchange, strong cation exchange, weak anion exchange, medium anion exchange, strong anion exchange and polystyrene/divinylbenzene polymers or copolymers thereof.

The stereochemistry can be denoted in several ways. For the amino acids often D/L is used for the alpha-position referring to the direction change of the optical rotation using polarized light. Stereochemically D corresponds to the stereodescriptor “R” and L corresponds to the stereodescriptor “S” for all of the compounds of the invention.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

D stands for Deuterium.

[¹⁸F]DFMT stands for (R)-2-Amino-3-(4-[¹⁸F]fluoromethoxy-phenyl)-propionic acid.

[¹⁸F]FMT stands for (S)-2-Amino-3-(4-[¹⁸F]fluoromethoxy-phenyl)-propionic acid.

The entire disclosure[s] of all applications, patents and publications, cited herein are incorporated by reference herein.

The term “purification” as employed herein has the objective to eliminate the excess of side product such as ¹⁸F-Fluorine and to concentrate and trap the reaction product. Purification is carried out by any method known to those in the art, suitable for radiotracer e.g. solid-phase-extraction cartridges or column.

The wording “automated and/or remote controlled device” refers to a device that is suitable for carrying out the radiosynthesis of a radiolabeled compound and maybe fully automated. The device comprises a reactor system, valves modules and a controller adapted to control the operation of said network.

The following examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Unless otherwise specified, when referring to the compounds of formula the present invention per se as well as to any pharmaceutical composition thereof the present invention includes all of the hydrates, salts, and complexes.

General Synthesis of F-18 Compounds

The radiofluorination reaction can be carried out, for example in a typical reaction vessel (e.g. Wheaton vial) which is known to someone skilled in the art or in a microreactor. The reaction can be heated by typical methods, e.g. oil bath, heating block or microwave. The radiofluorination reactions are carried out in dimethylformamide with potassium carbonate as base and “kryptofix” as crown-ether. But also other solvents can be used which are well known to experts. These possible conditions include, but are not limited to: dimethylsulfoxide and acetonitrile as solvent and tetraalkyl ammonium and tetraalkyl phosphonium carbonate as base. Water and/or alcohol can be involved in such a reaction as co-solvent. The radiofluorination reactions are conducted for one to 60 minutes. Preferred reaction times are five to 50 minutes. Further preferred reaction times are 10 to 40 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). The radiofluorination can be carried out in a “hot-cell” and/or by use of a module (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated or semi-automated synthesis.

The radiofluorination reaction can be carried out, for example in a typical reaction vessel (e.g. Wheaton vial) which is known to someone skilled in the art or in a microreactor. The reaction can be heated by typical methods, e.g. oil bath, heating block or microwave. The radiofluorination reactions are carried out in dimethylformamide with potassium carbonate as base and “kryptofix” as crown-ether. But also other solvents can be used which are well known to experts. These possible conditions include, but are not limited to: acetonitrile, dimethylsulfoxide, sulfolane, dichloromethane, tetrahydrofuran, tertiary alcohols and o-dichlorobenzene as solvent and alkali metal with and without a suitable alkali metal chelating crown ether, tetraalkyl ammonium and tetraalkyl phosphonium carbonate as base. Water and/or alcohol can be involved in such a reaction as co-solvent. The radiofluorination reactions are conducted for one to 60 minutes. Preferred reaction times are five to 50 minutes. Further preferred reaction times are 10 to 40 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). The radiofluorination can be carried out in a “hot-cell” and/or by use of a module (eview: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated or semi-automated synthesis.

General Synthesis of F-19 Compounds

Scheme 2 shows a method that can be used to synthesize of the racemic O-[¹⁹F]fluoromethyl tyrosine 4 starting from 1 using methods known to those skilled in the art. The synthesis of 2 from 1 is known in the literature (Liu et al., J. Med. Chem., 2004, 47, 1223-1233). The method for alkylating 2 to 3 is known for 0-fluoromethyltyrosine (J Labelled Compds. Radiopharm. 2003, 46, 555-566), similar methods can be applied here. Methods for hydrolyzing carboxylic acid protecting groups like methyl esters and amine protecting groups like t-butoxy carbonyl (Boc) groups are well precedented (Greene and Wuts, ‘Protecting Groups in Organic Syntheses’, third edition, pp. 369-453 and 494-653 respectively). The R and S isomers can be separated by methods known by those skilled in the art, i.e. chiral HPLC.

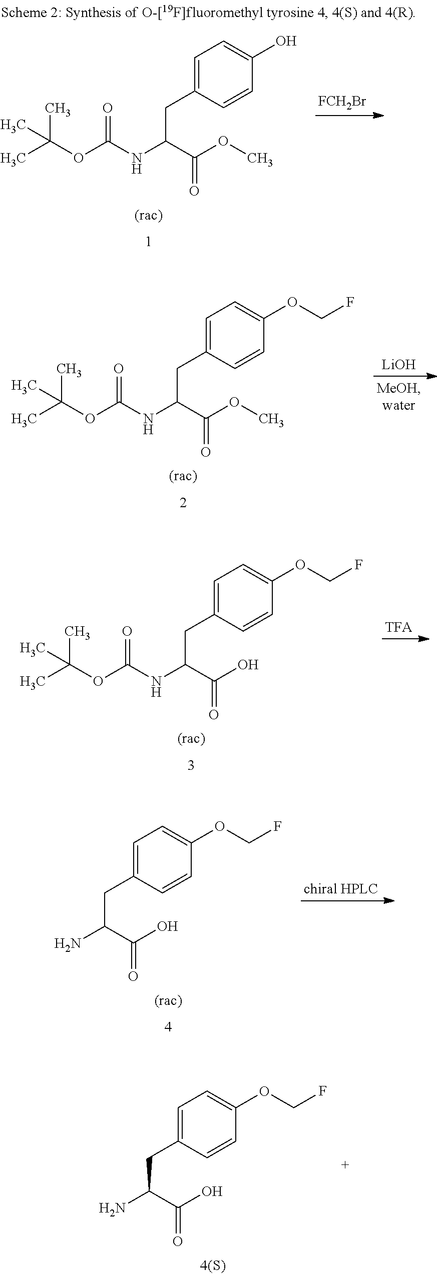

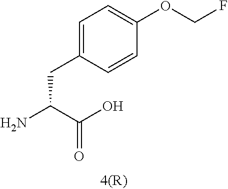 [SMILES: N[C@H](Cc1ccc(OCF)cc1)C(=O)O]

General Synthesis of F-18 Compounds

The ¹⁸F-compounds were synthesized by reaction of precursors of Formula II with [¹⁸F]Fluorine to give ¹⁸F labeled intermediates of Formula III which were then reacted with precursors of Formula IV to give the desired product of Formula I as shown in Scheme 3.

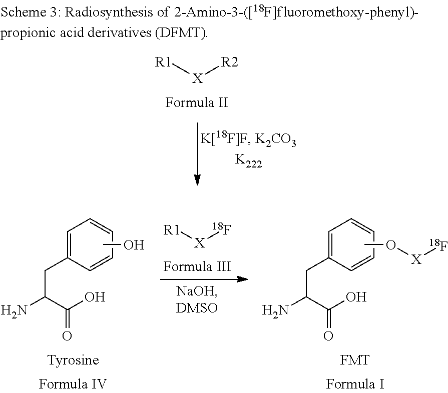

Preferably, the ¹⁸F-compounds were synthesized by reaction of precursors of Formula II (e.g. dibromomethane) with [¹⁸F]Fluorine to give ¹⁸F labeled intermediates of Formula III (e.g. bromo[¹⁸F]fluoromethane) which were then reacted with tyrosine precursors of Formula IV to give the desired product of Formula Ia as shown in Scheme 4.

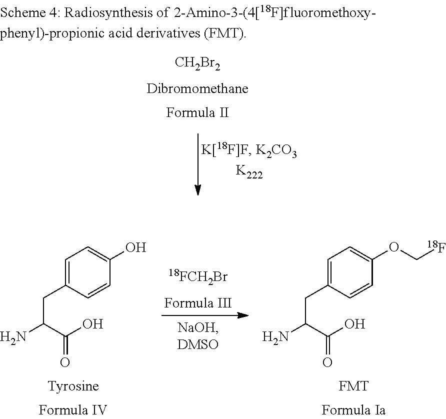

More preferably, the ¹⁸F-compounds were synthesized by reaction of dibromomethane with [¹⁸F]Fluorine isotope to give bromo[¹⁸F]fluoromethane which were then reacted with D-tyrosine to give the desired product DFMT as shown in Scheme 5.

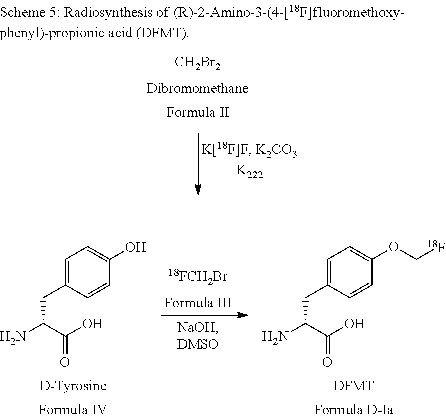

The purification of compounds of Formula I or Ia from the compounds of Formulae (II), (III),

(IV), reaction additives and organic solvents can be carried out using SPE methods instead of HPLC as outlined in Scheme 6.

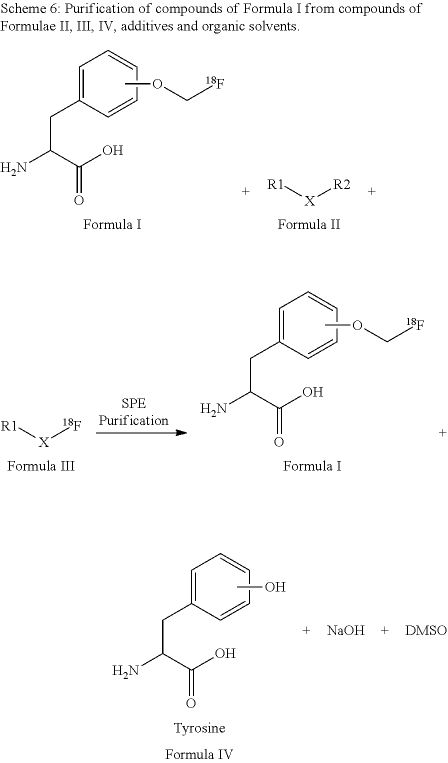

The purification of compounds of Formula D-Ia (i.e. DFMT) from the compounds of Formulae (II; i.e. dibromomethane), (III; i.e. bromo[¹⁸F]fluoromethane), (IV, i.e. tyrosine), reaction additives (i.e. sodium hydroxide) and organic solvents (i.e. DMSO) can be carried out using SPE methods instead of HPLC as outlined in Scheme 7.

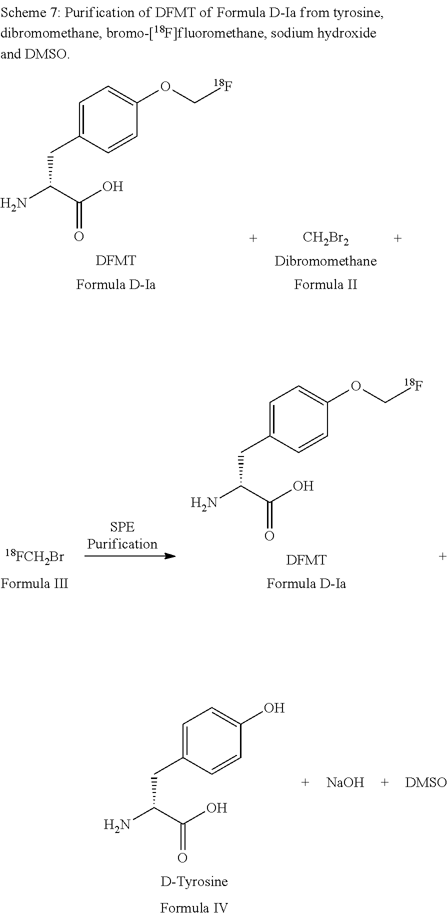

Experimental Section

Abbreviations

| | |
|---|---|
| C18E | C18 Plus Enviromental |
| DMSO | Dimethylsulfoxide |
| EtOH | Ethanol |
| GBq | GigaBequerel |
| HLB | Hydrophilic-Lipophilic Balance |
| HPLC | High Pressure Liquid Chromatography |
| K₂₂₂ | Kryptofix 2.2.2 |
| K₂CO₃ | Potassium carbonate |
| min | minute |
| MBq | MegaBequerel |
| QMA | Quaternary Methyl Ammonium |
| r.t. | room temperature |
| SCX | Strong Cation Exchange |
| SPE | Solid Phase Extraction |

General: All solvents and chemicals were obtained from commercial sources and used without further purification. Anhydrous solvents and inert atmosphere (nitrogen or argon) were used if not stated otherwise. The preceding table lists the abbreviations used in this paragraph and in the Examples sections as far as they are not explained within the text body.

The compounds and intermediates produced according to the methods of the invention may require purification, i.e. semi-preparative HPLC according to the preparative HPLC methods listed below.

### EXAMPLE 1

### HPLC Purification Method

### (R)-2-Amino-3-(4-[¹⁸F]fluoromethoxy-phenyl)-propionic acid ([¹⁸F]DFMT)

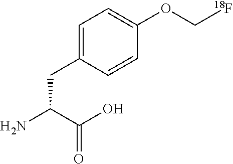 [SMILES: N[C@H](Cc1ccc(OC[18F])cc1)C(=O)O]

Typically [¹⁸F]Fluorine isotope was immobilized on a preconditioned QMA (Waters) cartridge (preconditioned by washing the cartridge with 5 ml 0.5M K₂CO₃ and 10 ml water), The [¹⁸F]Fluorine isotope was eluted using a solution of K₂CO₃ (2.7 mg) in 50 μl water and K₂₂₂ (15 mg) in 950 μl acetonitrile. This solution was dried at 120° C. with stirring under vacuum and with a nitrogen flow of 150 ml/min. Additional acetonitrile (1 ml) was added and the drying step was repeated. A solution of dibromomethane (CH₂Br₂; 100 μl) in acetonitrile (900 μl) was added and heated at 130° C. for 5 min. The reaction was cooled to 50° C. and the bromo[¹⁸F]fluoromethane was distilled at 50° C. with a nitrogen flow of 50 ml/min through 4 silica cartridges into a solution of D-tyrosine (3 mg), with 10% NaOH (13.5 μl) in DMSO (1 ml). This solution was heated at 110° C. for 5 min and then cooled to 40° C. The reaction mixture was purified by HPLC (Synergi Hydro RP 4μ 250×10 mm; 10% acetonitrile in water at pH 2; flow 5 ml/min). The product peak was collected, diluted with water (adjusted to pH 2 with HCl) and passed through a C18 Plus Environmental SPE. The SPE was washed with water (adjusted to pH 2 with HCl). The product was eluted with a 1:1 mixture of EtOH and water pH2 (3 ml). The yields of different runs are shown in Table 1 (n=≧3 runs).

### EXAMPLE 2

### SPE Purification Method Using SCX SPE Cartridges

### (R)-2-Amino-3-(4-[¹⁸F]fluoromethoxy-phenyl)-propionic acid ([¹⁸F]DFMT)

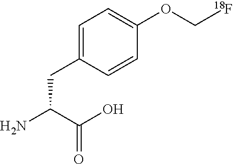 [SMILES: N[C@H](Cc1ccc(OC[18F])cc1)C(=O)O]

Typically [¹⁸F]Fluorine isotope was immobilized on a preconditioned QMA (Waters) cartridge (preconditioned by washing the cartridge with 5 ml 0.5M K₂CO₃ and 10 ml water), The [¹⁸F]Fluorine isotope was eluted using a solution of K₂CO₃ (2.7 mg) in 50 μl water and K₂₂₂ (15 mg) in 950 μl acetonitrile. This solution was dried at 120° C. with stirring under vacuum and with a nitrogen flow of 150 ml/min. Additional acetonitrile (1 ml) was added and the drying step was repeated. A solution of dibromomethane (CH₂Br₂; 100 μl) in acetonitrile (900 μl) was added and heated at 130° C. for 5 min. The reaction was cooled to 50° C. and the bromo[¹⁸F]fluoromethane was distilled at 50° C. with a nitrogen flow of 50 ml/min through 4 silica cartridges into a solution of D-tyrosine (3 mg), with 10% NaOH (13.5 μl) in DMSO (1 ml). This solution was heated at 110° C. for 5 min and then cooled to 40° C. The reaction mixture was diluted with 20 mL pH2 water (pH adjusted with 1M HCl) and passed through an unconditioned SCX cartridge (Varian, 1000 mg, 45 mm), this cartridge was washed with 30 mL pH2 water (pH adjusted with 1M HCl) and the product was eluted with 10-20 mL saline solution (7 g Na₂HPO₄ and 6 g NaCl in 1 liter water). The yields of different runs are shown in Table 1 (n=≧3 runs).

SCX cartridge is silica ion exchange solid phase extraction.

### EXAMPLE 3

### SPE Purification Method Using HLB SPE Cartridges

### (R)-2-Amino-3-(4-[¹⁸F]fluoromethoxy-phenyl)-propionic acid ([¹⁸F]DFMT)

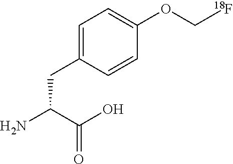 [SMILES: N[C@H](Cc1ccc(OC[18F])cc1)C(=O)O]

Typically [¹⁸F]Fluorine isotope was immobilized on a preconditioned QMA (Waters) cartridge (preconditioned by washing the cartridge with 5 ml 0.5M K₂CO₃ and 10 ml water), The [¹⁸F]Fluorine isotope was eluted using a solution of K₂CO₃ (2.7 mg) in 50 μl water and K₂₂₂ (15 mg) in 950 μl acetonitrile. This solution was dried at 120° C. with stirring under vacuum and with a nitrogen flow of 150 ml/min. Additional acetonitrile (1 ml) was added and the drying step was repeated. A solution of dibromomethane (CH₂Br₂; 100 μl) in acetonitrile (900 μl) was added and heated at 130° C. for 5 min. The reaction was cooled to 50° C. and the bromo[¹⁸F]fluoromethane was distilled at 50° C. with a nitrogen flow of 50 ml/min through 4 silica cartridges into a solution of D-tyrosine (3 mg), with 10% NaOH (13.5 μl) in DMSO (1 ml). This solution was heated at 110° C. for 5 min and then cooled to 40° C. The reaction mixture was diluted with 20 mL pH2 water (pH adjusted with 1M HCl) and passed through an a series of four HLB cartridges (Waters OASIS HLB Plus LP, preconditioned by washing the cartridge with 5 ml ethanol and 10 ml water), this cartridge was washed with 40 mL pH2 water (pH adjusted with 1M HCl) and the product was eluted with a mixture 3 mL EtOH and 3 mL pH2 water. The yields of different runs are shown in Table 1 (n=≧3 runs).

Waters OASIS HLB Plus LP cartridge is a polymeric water-wettable reversed-phase sorbent.

### EXAMPLE 4

### SPE Purification Method Using SCX SPE Cartridges

### (S)-2-Amino-3-(4-[¹⁸F]fluoromethoxy-phenyl)-propionic acid ([¹⁸F]FMT)

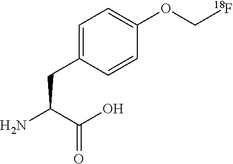 [SMILES: N[C@@H](Cc1ccc(OC[18F])cc1)C(=O)O]

Typically [¹⁸F]Fluorine isotope was immobilized on a preconditioned QMA (Waters) cartridge (preconditioned by washing the cartridge with 5 ml 0.5M K₂CO₃ and 10 ml water), The [¹⁸F]Fluorine isotope was eluted using a solution of K₂CO₃ (2.7 mg) in 50 μl water and K₂₂₂ (15 mg) in 950 μl acetonitrile. This solution was dried at 120° C. with stirring under vacuum and with a nitrogen flow of 150 ml/min. Additional acetonitrile (1 ml) was added and the drying step was repeated. A solution of dibromomethane (CH₂Br₂; 100 μl) in acetonitrile (900 μl) was added and heated at 130° C. for 5 min. The reaction was cooled to 50° C. and the bromo[¹⁸F]fluoromethane was distilled at 50° C. with a nitrogen flow of 50 ml/min through 4 silica cartridges into a solution of D-tyrosine (3 mg), with 10% NaOH (13.5 μl) in DMSO (1 ml). This solution was heated at 110° C. for 5 min and then cooled to 40° C. The reaction mixture was diluted with 20 mL pH2 water (pH adjusted with 1M HCl) and passed through an unconditioned SCX cartridge (Varian, 1000 mg, 45 mm), this cartridge was washed with 30 mL pH2 water (pH adjusted with 1M HCl) and the product was eluted with 10-20 mL saline. The yields of different runs are shown in Table 1 (n=1 run).

TABLE 1
Summary of the radiosyntheses of [¹⁸F]fluoromethyl tyrosine derivatives

| | | | | | Specific | |
|---|---|---|---|---|---|---|
| | | Purification | Radiochemical | Standard | Activity | Time |
| | Run | Description | Yield (d.c.) | Deviation | (GBq/μmol) | (min) |
| Example 1 | 1 | HPLC + | 13.4% | 12.0 ± 1.9% | 17.2 | 73 |
| | | C18E | | | | |
| | 2 | HPLC + | 13.4% | | 67.9 | 71 |
| | | C18E | | | | |
| | 3 | HPLC + | 10.9% | | 53.7 | 71 |
| | | C18E | | | | |
| | 4 | HPLC + | 10.4% | | 64 | 70 |
| | | C18E | | | | |
| Example 2 | 1 | SCX only | 18.5% | 19.6 ± 1.2% | 78.3 | 65 |
| | 2 | SCX only | 19.5% | | >55 | 58 |
| | 3 | SCX only | 20.8% | | >135 | 64 |
| Example 3 | 1 | 4 x HLB only | 14.5% | 13.9 ± 2.1% | >68 | 65 |
| | 2 | 4 x HLB only | 15.7% | | >72 | 58 |
| | 3 | 4 x HLB only | 11.6% | | >56 | 56 |
| Example 4 | 1 | SCX only | 21.2% | na | 81.7 | 95 |

(d.c.)

1.-12. (canceled)

13. A method for the purification of compound of formula (I) 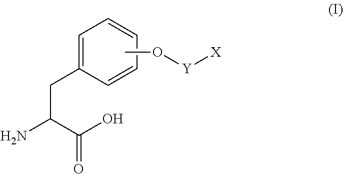
wherein
X is a Fluorine atom (F);
Y is CH₂, CHD, or CD₂;
D stands for Deuterium; and
single isomers, and enantiomers, mixtures thereof and pharmaceutically acceptable salts thereof;
comprising the step of
Purification of compound of formula (I) by solid-phase-extraction (SPE) conducted with
one SPE cartridge wherein the solid phase of the cartridge is a cation exchange resin or
two to four SPE cartridges wherein the solid phase of the cartridge is a polymer based resin.

14. The method according to claim 13 wherein the step of purification is conducted with one SCX (Strong Cationic Exchanger) SPE cartridge.

15. The method according to claim 14 wherein the SCX SPE cartridge contains the cation exchange resin from the range of about 0.1 to about 2 g.

16. The method according to claim 13 wherein the step of purification is conducted with two to four HLB (Hydrophilic-Lipophilic Balance) SPE cartridges.

17. The method according to claim 16 wherein the HLB SPE cartridge contains the polymeric water-wettable reversed-phase sorbent from the range of about 0.1 to about 2 g.

18. The method according to claim 13 wherein X is a [¹⁸F] Fluorine isotope.

19. The method according to claim 13 wherein Y is CH₂ or CD₂.

20. The method according to claim 13 wherein the compound of formula (I) is compound of formula (I-D) or (I-L). 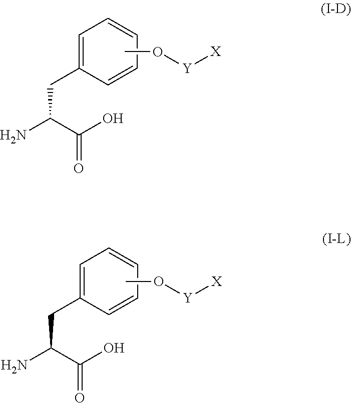
and X and Y as defined in claim 13.

21. The method according to claim 13 wherein compound of formula (I) is selected from [¹⁸F]DFMT standing for (R)-2-Amino-3-(4-[¹⁸F]fluoromethoxy-phenyl)-propionic acid, DFMT standing for (R)-2-Amino-3-(4-fluoromethoxy-phenyl)-propionic acid, [¹⁸F]Deuterio-DFMT standing for (R)-2-Amino-3-(4-[¹⁸F]fluorodideuteriomethoxy-phenyl)-propionic acid, Deuterio-DFMT standing for (R)-2-Amino-3-(4-fluorodideuteriomethoxy-phenyl)-propionic acid, [¹⁸F]FMT standing for (S)-2-Amino-3-([¹⁸F]fluoromethoxy-phenyl)-propionic acid, FMT standing for (S)-2-Amino-3-(4-fluoromethoxy-phenyl)-propionic acid, [¹⁸F]Deuterio-FMT standing for (S)-2-Amino-3-(4-[¹⁸F]fluorodideuteriomethoxy-phenyl)-propionic acid and Deuterio-FMT standing for (S)-2-Amino-3-(4-fluorodideuteriomethoxy-phenyl)-propionic acid.

22. The method according to claim 21 wherein compound of formula (I) is [¹⁸F]DFMT standing for (R)-2-Amino-3-(4-[¹⁸F]fluoromethoxy-phenyl)-propionic acid.

23. A method for obtaining a purified compound of formula (I) comprising the step of
indirect fluoro-labeling or direct fluoro-labeling step for obtaining compound of formula (I) 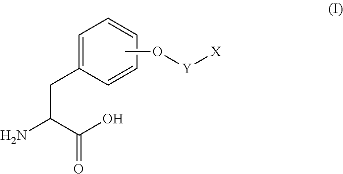
wherein
X is a Fluorine atom (F);
Y is CH₂, CHD, or CD₂; and
D stands for Deuterium
and
Purification of compound of formula (I) by solid-phase-extraction (SPE) conducted with
one SPE cartridge wherein the solid phase of the cartridge is a cation exchange resin or
two to four SPE cartridges wherein the solid phase of the cartridge is a polymer based resin.

24. A composition comprising compounds of the formula (I) obtainable from the methods of claim 13.
